(19) United States
(12) Patent Application Publication
(10) Pub. No.: US 2026/0033734 A1
(43) Pub. Date: Feb. 5, 2026

(54) LINEARLY CONTINUOUS PHASED WINDOWING METHOD IN FMCW SYSTEM FOR DETECTION OF BIO-SIGNALS

(71) Applicant: Jin Myung KIM, Buchon-si (KR)

(72) Inventor: Jin Myung KIM, Buchon-si (KR)

(21) Appl. No.: 19/348,789

(22) Filed: Oct. 3, 2025

Related U.S. Application Data
(63) Continuation of application No. PCT/KR2024/005265, filed on Apr. 18, 2024.

(30) Foreign Application Priority Data
Apr. 21, 2023 (KR) 10-2023-0052369

(51) Int. Cl. A61B 5/05; A61B 5/00; G01S 7/35

(52) U.S. Cl. CPC A61B 5/05; A61B 5/7257; G01S 7/356

(57) ABSTRACT

Provided is a linearly continuous phased windowing method in an FMCW system for detection of bio-signals, which improves the performance and stability of detecting fine movement information of a target by applying a linear and highly continuous phased windowing method to an FFT preprocessing process in the FMCW system for detection of bio-signals. The present disclosure has an advantage of high detection performance and stability of a fine movement of a target in an FMCW system for detection of bio-signals.

### TECHNICAL FIELD

The present disclosure relates to a linearly continuous phased windowing method in an FMCW system for detection of bio-signals, which improves the performance and stability of detecting fine movement information of a target by applying a linear and highly continuous phased windowing method to an FFT preprocessing process in the FMCW system for detection of bio-signals.

### BACKGROUND ART

In general, a frequency modulated continuous wave (FMCW) system used in radar, laser, or lidar receives a complex beat signal and processes the signal in an order of N-point signal analog/digital converter (ADC), N-point windowing, N-point fast Fourier transformer (FFT), and magnitude.

In FIG. 1, a general FMCW system 10 that detects a target 20 generates a frequency modulated signal using a frequency modulator 100. Furthermore, the generated signal is transmitted to a transmitting antenna Tx 101. The transmitted signal is reflected by the target 20 and received by a receiving antenna Rx 102. The signal received by the receiving antenna Rx 102 is amplified through an amplifier 103. Furthermore, the amplified signal and the frequency modulated signal from the frequency modulator 100 are applied to a first mixer 104 to generate a complex bit signal generated from a difference between the signals, which is converted into an in-phase bit signal r_{I}(t) or a real bit signal and output. In addition, the signal amplified by the amplifier 103 and the signal frequency-modulated by the frequency modulator 100 are applied to a second mixer 105 as a signal with a phase shift of 90 degrees through a phase shifter 106 and converted into a quadrature-phase bit signal r_{Q}(t) or an imaginary bit signal as a complex bit signal generated from a difference between signals and output.

Moreover, the FMCW system 10 may detect information such as the position, speed, and phase of the target 20 by using the in-phase bit signal r_{I}(t) and the quadrature-phase bit signal r_{Q}(t) as complex bit signals, and for this purpose, processing as shown in FIG. 2 is required. The complex bit signals r_{I}(t)) and r_{Q}(t) are converted into digital signals through respective analog/digital converters 200, 201. The digital real bit signal r_{I}(n) converted by the first analog/digital converter 200 is stored in N first buffers 202. At the same time, a digital imaginary bit signal r_{Q}(n) converted by the second analog/digital converter 201 is stored in N second buffers 203. Furthermore, the signals stored in the first buffer 202 and the second buffer 203 are multiplied by a windowing coefficient w(n) 204 at a first multiplier 205 and a second multiplier 206, and N real windowed signals y_{I}(n) and N imaginary windowed signals (y)_{Q}(n) are stored in a third buffer 207 and a fourth buffer 208, respectively. In addition, the signal of the third buffer 207 and the signal of the fourth buffer 208 are input to a fast Fourier transformer (FFT) 209, and N fast Fourier transformed real part (FFT Real) signals Y₁(k) and N fast Fourier transformed imaginary part (FFT Imaginary signals Y_{Q}(k) that are fast Fourier transformed in a fast Fourier transformer (FFT) 209 are stored in a fifth buffer 210 and a sixth buffer 211, respectively.

Y(k) is commonly referred to as a profile, and a peak point of a profile magnitude indicates a position of the target 20 around the FMCW system 10. Here, the role of the fast Fourier transformer (FFT) 209 is to generate a profile, and the role of windowing is to improve the quality of the profile.

In addition, in order to detect not only the position of the target but also its fine movement using the FMCW system, ∠Y(k), that is, the profile phase, must be used together with the profile magnitude |Y(k)|. Therefore, the position of the target is detected with |Y(k)|, and then a fine movement of the target is detected with ∠Y(k).

The windowing reduces a sidelobe of a peak rod in |Y(k)| so as to improve the position detection performance of the target. However, the problem of conventional windowing causes severe discontinuous distortion in ∠Y(k), making it difficult to detect a fine movement. As a result, there is a problem in that the detection of fine movement information of the FMCW system is very unstable.

Research using a recent FMCW system to detect position information of living things such as humans or animals and bio-signal information such as breathing or heartbeat is actively being conducted as shown in FIG. 3. Bio-signal information usually has minute movements and is periodic, which increases the instability of detection of bio-signal information and degrades performance when using a conventional FMCW system that has a problem in detecting fine movement information as shown in FIG. 2. Therefore, in order to solve the problems of the FMCW system, it is necessary to develop a new windowing that improves the quality of the |Y(k)| and at the same time does not cause discontinuous distortion in the ∠Y(k).

As a prior art related to the present disclosure, Patent Document 1 discloses a radar signal processing method using a nonlinear frequency modulation waveform, including (a) generating and storing a delta frequency value using a window function; (b) outputting the stored delta frequency value according to a preset counter timing; and (c) receiving the output delta frequency value to transmit it as a nonlinear frequency modulation (NLFM) signal waveform using a direct digital synthesis method.

### DISCLOSURE OF INVENTION

### Technical Problem

The present disclosure is intended to solve the foregoing problems, and an aspect of the present disclosure is to present a linear and highly continuous phased windowing method in an FMCW system for detection of bio-signals so as to improve the performance and stability of fine movement information detection.

In addition, another aspect of the present disclosure is to apply windowing for FFT pre-processing in an FMCW system used in radar, laser, lidar, or the like so as to detect the fine movement of a target.

### Technical Solution

In order to achieve the foregoing objectives, a linearly continuous phased windowing method in an FMCW system for detection of bio-signals may include (a) computing a profile W(k) by passing a complex window signal w(n) derived from the FMCW system through a fast Fourier transformer (FFT) to calculate an FFT(w(n)), and deriving a phase value ∠W(k) from the computed profile W(k); (b) detecting a plurality of discontinuous points (k_{d} ld=0, . . . D−1) from the phase value ∠W(k) and then multiplying the corresponding profile W(k) by a frequency variation e^{jπ} to compute a new profile W_{new}(k); (c) computing IFFT(^{w̅}_{new}(n)) by passing the new profile W_{new}(k) through an inverse fast Fourier transformer (IFFT) to calculate ^{w̅}_{new}(n); and (d) computing real(w_{new}(n)) of a real part from the calculated ^{w̅}_{new}(n) to calculate w_{new}(n).

In addition, in the present disclosure, in a linearly continuous phased windowing method in an FMCW system for detection of bio-signals, a first half value and a second half value of N window values w(n) derived from the FMCW system may be exchanged with each other to calculate a new window value w_{new}(n).

In addition, in the present disclosure, a linearly continuous phased windowing method in an FMCW system for detection of bio-signals may include (a) computing a profile W(k) by passing a complex window signal w(n) derived from the FMCW system through a fast Fourier transformer (FFT) to calculate an FFT(w(n)), and deriving a magnitude value |W(k)| from the computed profile W(k); (b) computing a profile W(k) by passing a complex window signal w(n) derived from the FMCW system through a fast Fourier transformer (FFT) to calculate an FFT(w(n)), and deriving a phase value ∠W(k) from the computed profile W(k); (c) detecting a plurality of discontinuous points (k_{d} ld=0, . . . D−1) from the phase value ∠W(k) and then multiplying the corresponding profile W(k) by a frequency variation e^{jπ} to compute a new profile W_{new}(k); (d) computing IFFT(^{w̅}_{new}(n)) by passing the new profile (W_{new}(k)) through an inverse fast Fourier transformer (IFFT) to calculate ^{w̅}_{new}(n); and (e) computing real(w_{new}(n)) of a real part from the calculated ^{w̅}_{new}(n) to calculate w_{new}(n).

### Advantageous Effects

According to the present disclosure, in an FMCW system for detection of bio-signals, a linear and highly continuous phased windowing method may be applied to a FFT preprocessing process, thereby having an advantage of improving the performance and stability of detecting fine movement information of bio-signals such as respiration or heart rate of a target.

### BRIEF DESCRIPTION OF THE DRAWINGS

FIG. 1 is a block diagram showing a general FMCW system for detection of bio-signals.

FIG. 2 is a block diagram showing an FMCW system that calculates a bit signal.

FIG. 3 is a conceptual diagram for detecting bio-signal information of living things using a general FMCW system.

FIG. 4A is a graph showing a time domain, frequency magnitude, and frequency phase according to a Hamming window method in an FMCW system.

FIG. 4B is a graph showing a time domain, frequency magnitude, and frequency phase according to a Blackman window method in an FMCW system.

FIG. 5 is a graph showing the characteristics of |Y(k)| and ∠Y(k) for detecting a target at a specific position according to a Hamming window method in an FMCW system.

FIG. 6 is a graph showing the characteristics of |Y(k)| and ∠Y(k) for detecting a target at a specific position according to a Blackman window method in an FMCW system.

FIG. 7 is a graph showing the characteristics of |Y(k)| and ∠Y(k) newly derived from an FMCW system based on an Hamming window method using linearly continuous phased windowing in an FMCW system for detection of bio-signals according to the present disclosure.

FIG. 8 is a table showing values of stability derived by a linearly continuous phased windowing method in an FMCW system for detection of bio-signals according to the present disclosure compared to a conventional windowing method.

FIG. 9 is a flowchart showing a method of deriving a linearly continuous phased window value in an FMCW system for detection of bio-signals according to an embodiment of the present disclosure.

FIG. 10 is a graph showing a result of deriving a new window value from a conventional Hamming window value by applying a linearly continuous phased windowing method in an FMCW system for detection of bio-signals according to the present disclosure.

FIG. 11 is a schematic diagram showing a method of deriving a first half value and a second half value from N arbitrary window values by exchanging with each other in a linearly continuous phased windowing method in an FMCW system for detection of bio-signals according to another embodiment of the present disclosure.

FIG. 12 is a graph showing a result of deriving a first half value and a second half value from N arbitrary window values by exchanging them each other in a linearly continuous phased windowing method in an FMCW system for detection of bio-signals according to the present disclosure.

FIG. 13 is a graph showing the characteristics of |Y(k)| and ∠Y(k) newly derived from an FMCW system based on a Blackman window using linearly continuous phased windowing in an FMCW system for detection of bio-signals according to the present disclosure.

FIG. 14A is a block diagram showing a method of deriving |Y(k)| and ∠Y(k) using a linearly continuous phased windowing method in an FMCW system for detection of bio-signals according to another embodiment of the present disclosure.

FIG. 14B is a block diagram showing a linearly continuous phased windowing method in an FMCW system for detection of bio-signals, in which |Y(k)| is derived by using a conventional window method, and ∠Y(k) is derived by using a window method of the present disclosure according to another embodiment of the present disclosure.

FIGS. 15A-15C are graphs comparing the detection performance of a fine movement of a target detected by a linearly continuous phased windowing method in an FMCW system for detection of bio-signals according to the present disclosure with that of a fine movement of a target detected by a conventional Blackman window-based FMCW system.

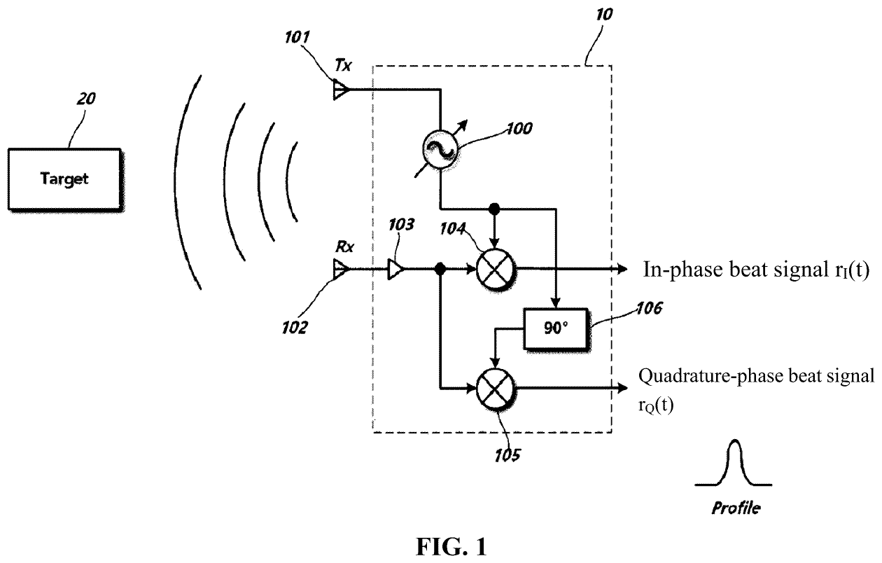

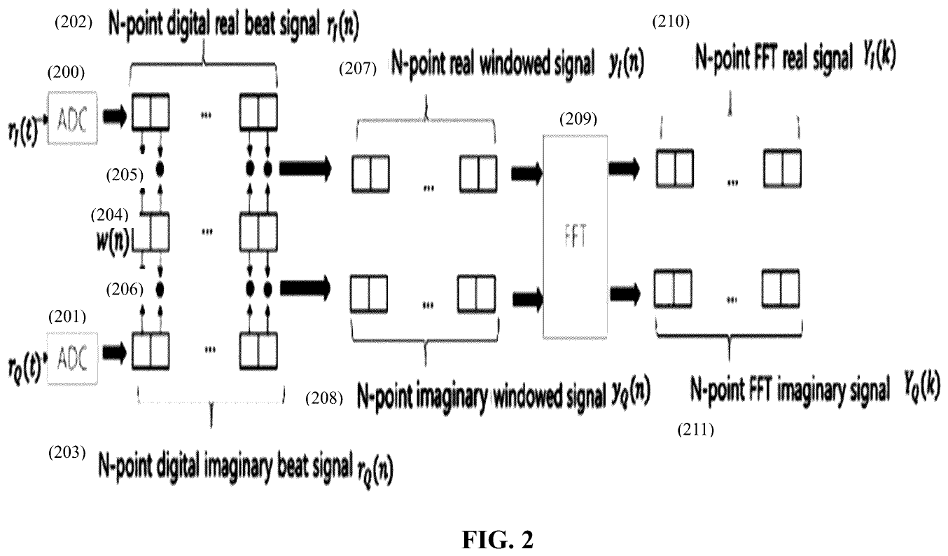

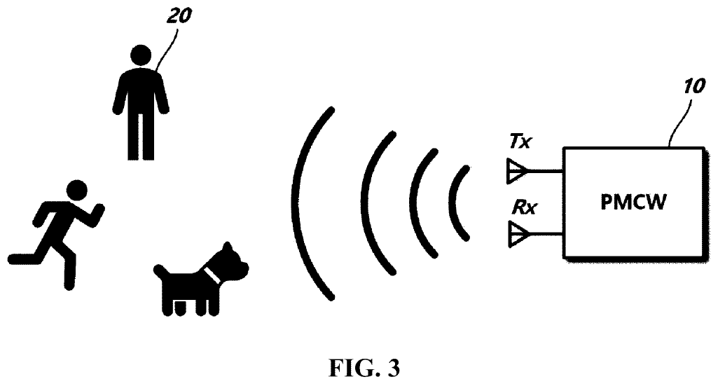

### BEST MODE FOR CARRYING OUT THE INVENTION

Hereinafter, an embodiment of a linearly continuous phased windowing method in an FMCW system for detection of bio-signals according to the present disclosure will be described in detail with reference to the accompanying drawings.

First, in order to describe a window, a Hamming window method and a Blackman window method, which are most commonly used among conventional windows, will be described.

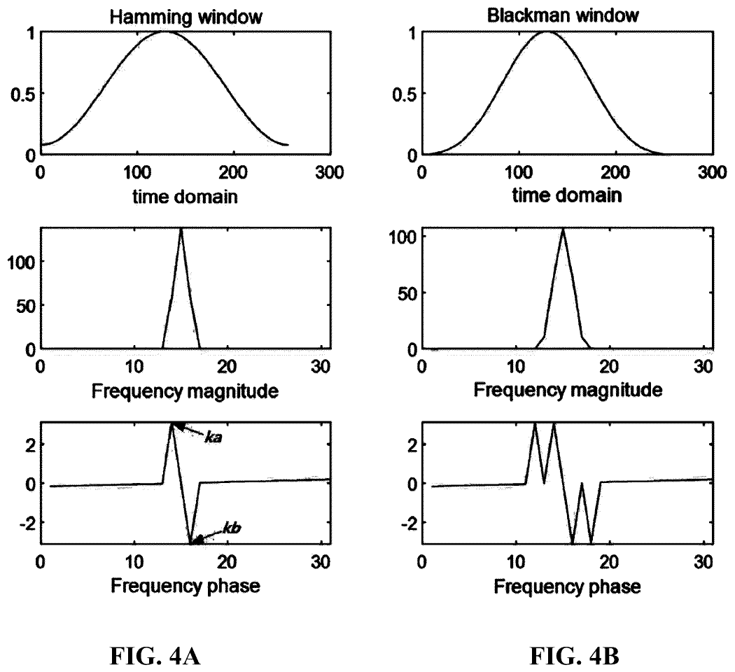

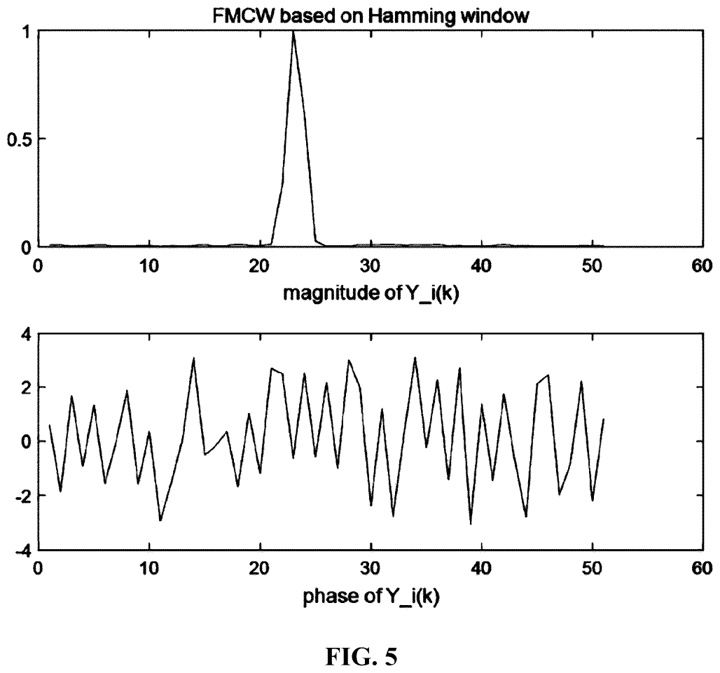

Referring to time domain and frequency domain signals as a Hamming window method in FIG. 4A and a Blackman window method in FIG. 4B, respectively, it can be seen that a phase of the frequency domain signal of each window exhibits a discontinuous characteristic. Furthermore, a graph in FIG. 5 shows a magnitude value |Y(k)| and a phase value ∠Y(k) in a FMCW system that detects a target at a specific position using the Hamming window method. Here, a signal to noise ratio (SNR) is set to 20 dB. Looking at |Y(k)|, the Hamming window method has no problem in finding a peak point, but it can be seen that ∠Y(k) is very noisy and less continuous, making it difficult to detect a phase near the peak point.

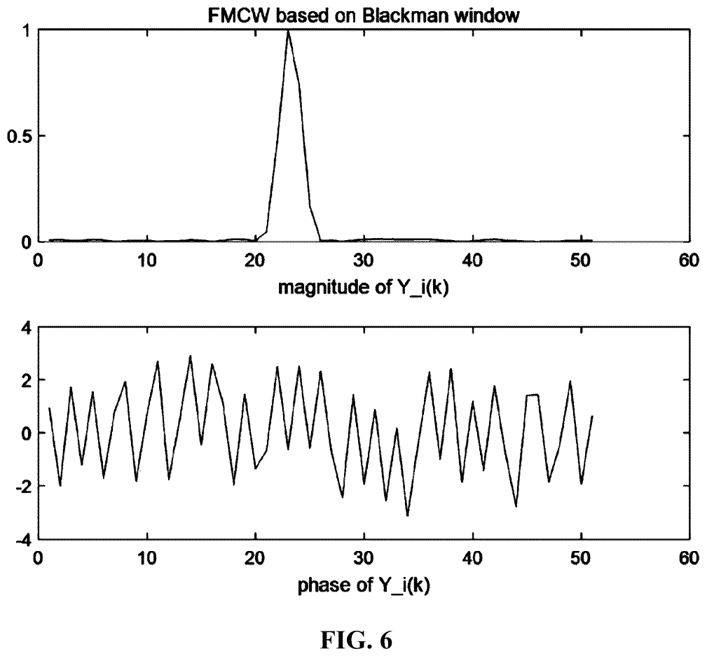

In addition, the Blackman window method in FIG. 6, like the Hamming window method, has difficulty in detecting a phase near the peak point.

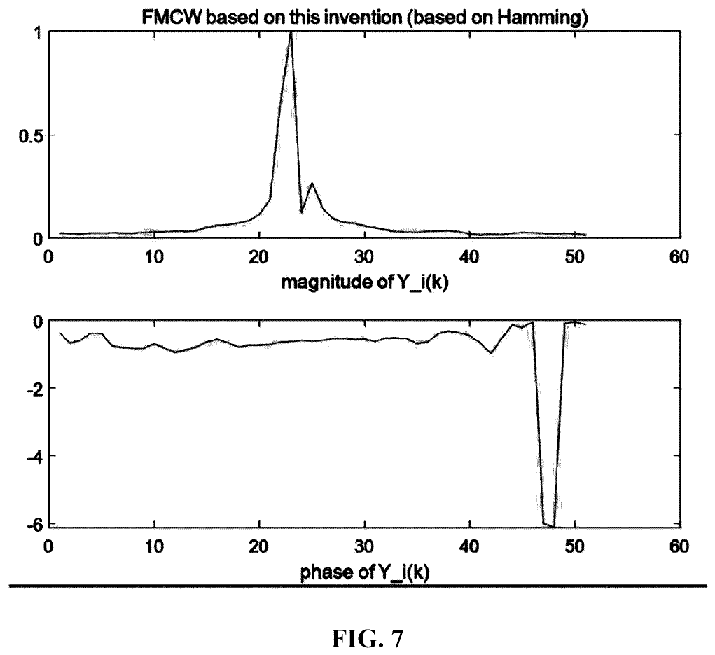

in FIG. 7, referring to a graph showing the results of |Y(k)| and ∠Y(k) in an FMCW system using a linear and highly continuous phased windowing method proposed in the present disclosure, it can be seen that, unlike the graphs showing the results of FIGS. 5 and 6 using conventional windowing methods, the noise characteristics of ∠Y(k) are significantly reduced and the linearity and continuity are improved. Due to this, a fluctuation range of phase value ∠Y(k) detected near a peak of |Y(k)| may be greatly reduced. The reason this is important is that the peak detection of |Y(k)| often varies over a range of □1 at a peak position. This may also cause a peak detection error due to noise added to |Y(k)|, and often occur when a fine movement fluctuates between integers k and k+1 or between k−1 and k due to a limited position resolution of |Y(k)|. Therefore, when the fluctuation range of the phase value near the peak of |Y(k)| is large, a fine movement cannot be properly detected.

For convenience, one indicator may be defined as in Equation 1 below for comparing the phase value fluctuation ranges or phase stabilities. That is, it indicates a degree of phase value fluctuation range around the peak point, and the larger the value, the higher the stability.

$$S = {\sum }_{( I = - L )}^{L} {\vert ∠ Y ( {k}_{0 + l} ) - ∠ Y ( {k}_{0} ) \vert}^{- 1} \qquad [ Equation 1 ]$$

Here, k₀ is an index value in which |Y(k₀)| is a peak.

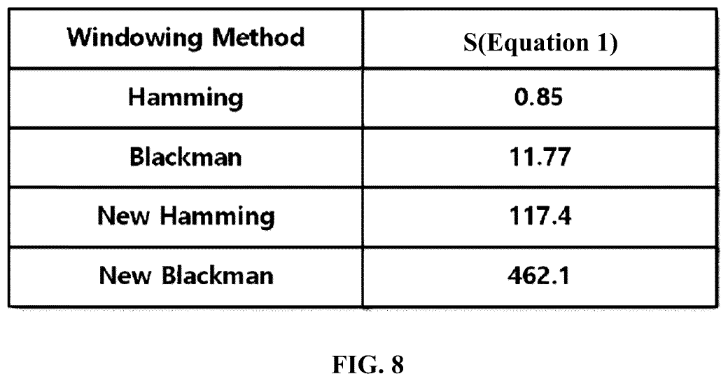

Accordingly, in FIG. 8, as a result of comparing stability for each windowing method, it can be seen that the stability of the windowing method of the present disclosure is much higher than that of the conventional windowing method, and thus stability can be secured.

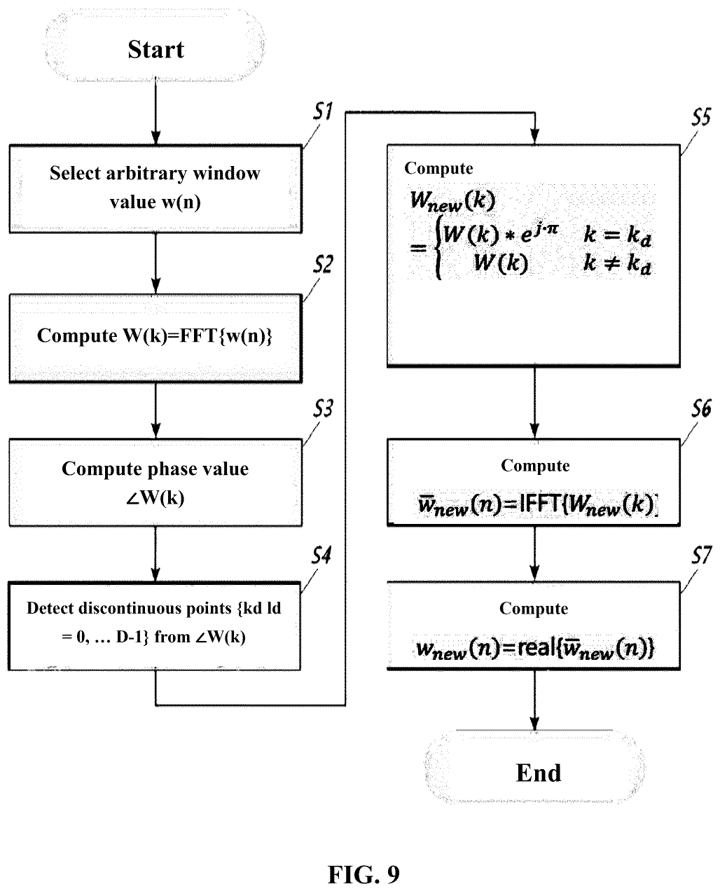

FIG. 9 is a method of deriving a window value having a linear and highly stable phased window value presented in the present disclosure. According to this method, a linear and highly stable phased window value may be derived from an arbitrary window value.

First, a complex window signal w(n) is derived by windowing in a conventional FMCW system (S1). Furthermore, the derived complex window signal w(n) is passed through a fast Fourier transformer (FFT) to calculate FFT(w(n)) and compute a profile W(k) (S2). A phase value ∠W(k) is derived from the calculated profile W(k) (S3). This will be a graph of the phase value ∠W(k) in FIGS. 4A-4B.

Next, a plurality of discontinuous points (k_{d} ld=0, . . . D−1) are detected from the phase value ∠W(k) (S4). For example, there are two discontinuous points in the Hamming window method in FIG. 4A, and four discontinuous points in the Blackman window method in FIG. 4B. In the present disclosure, it is assumed that there are two ka and kb according to the Hamming window method. Furthermore, a new profile W_{new}(k) is computed by multiplying the corresponding profile W(k) by a frequency variation e^{jπ} (S5). That is, W_{new}(kₐ)=W(kₐ)*e^{jπ}, W_{new}(k_{b})=W(k_{b})*e^{jπ} is multiplied, and otherwise leave it as it is, W_{new}(kₒₜₕₑᵣₛ)=W(kₒₜₕₑᵣₛ).

In addition, the new profile W_{new}(k) is passed through an inverse fast Fourier transformer (IFFT) to compute IFFT(^{w̅}_{new}(n)) so as to calculate ^{w̅}_{new}(n) (S6). Then, real(w_{new}(n)) of a real part is computed from the calculated ^{w̅}_{new}(n) to derive a new window value w_{new}(n) (S7).

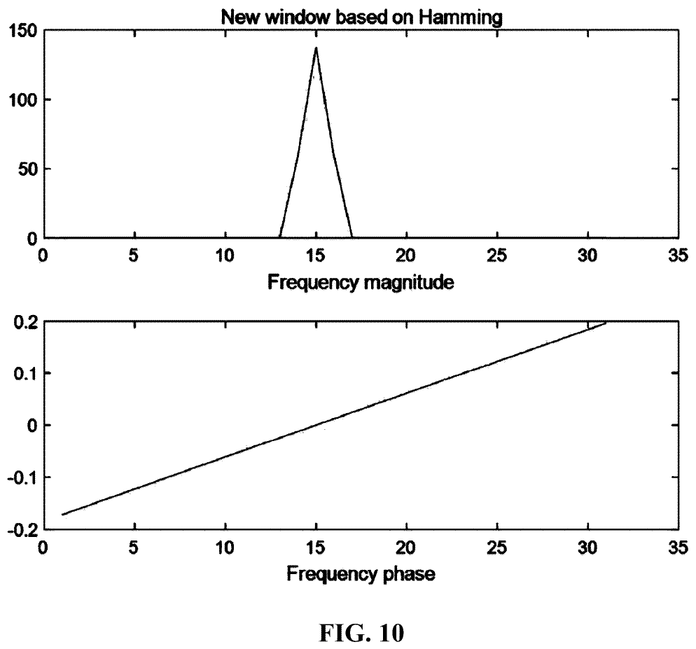

Therefore, a method of deriving a window value according to the present disclosure may be used to derive a new window value w_{new}(n) from a window value calculated in the conventional Hamming window method. As a result, in the characteristic graph of FIG. 10, more improved phase characteristics may be confirmed.

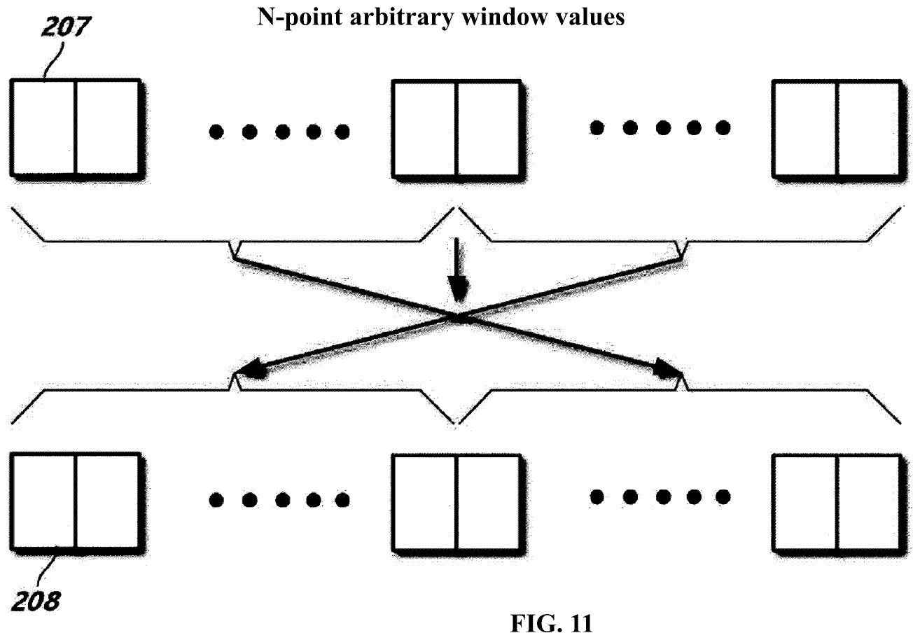

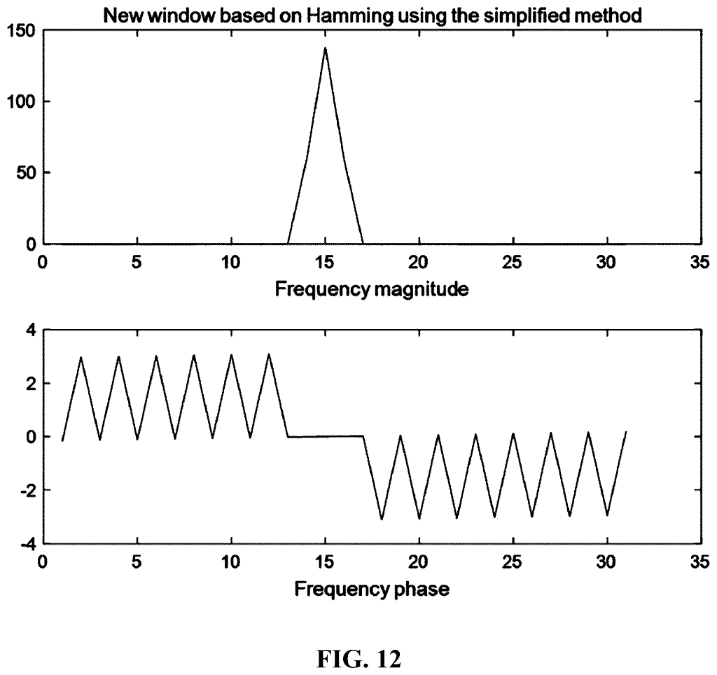

Next, FIG. 11 shows another embodiment of the present disclosure, in which a new window value can be derived more simply than in the above embodiment. That is, in FIG. 11, when there are N (N-point) arbitrary window values set, a new window value may be simply calculated by exchanging a first half value and a second half value with each other. The calculated magnitude characteristic is similar to the magnitude of FIG. 10, and the phase characteristic has a horizontal section in a section in which the magnitude is greater than 0, and in an index area of approximately 13th to 17th. That is, it means that windowing preserves the phase of the original signal, which is consistent with the phase characteristic having linear continuity. Therefore, when deriving a new window value, either the method presented in FIG. 9 or the method presented in FIG. 11 may be used. Moreover, in the present disclosure, a method of calculating a new window value by exchanging a first half value and a second half value with each other from N window values may be selectively applied to the Blackman window method or the Hamming window method to generate a new window value.

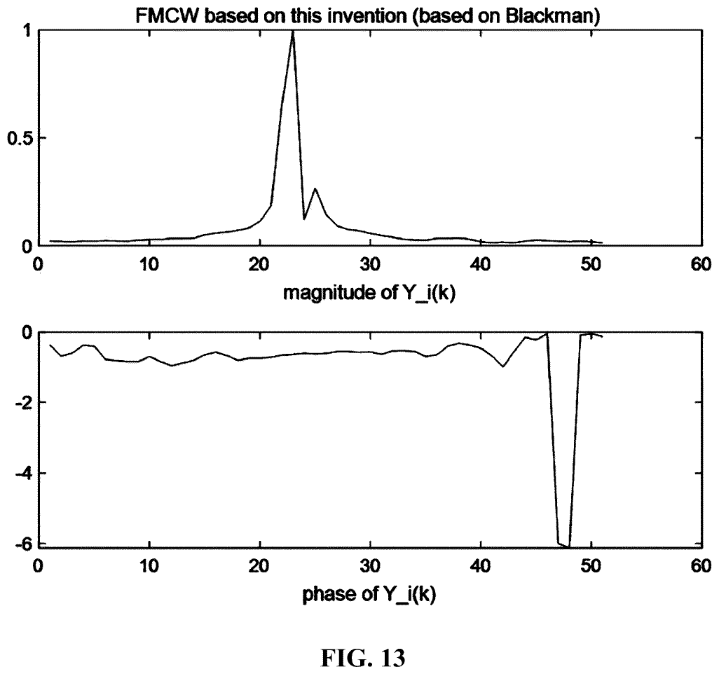

In addition, in the present disclosure, it can be seen that the phase characteristics of the window-based FMCW system of the present disclosure are excellent in both the graph showing |Y(k)| and ∠Y(k) of the new w_{hamming.new}(n)-based FMCW system of the present disclosure from the Hamming window-based FMCW system of FIG. 7 and the graph showing |Y(k)| and ∠Y(k) of the new w_{black.new}(n)-based FMCW system of the present disclosure from the Blackman window-based FMCW system of FIG. 13.

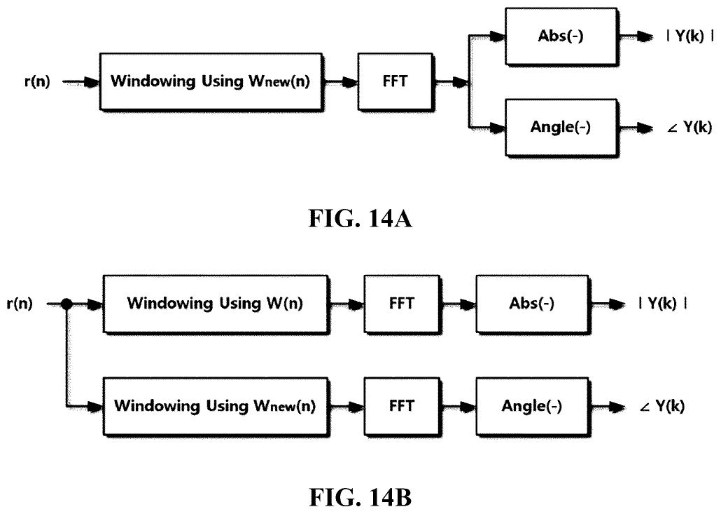

In addition, as a still another embodiment of the present disclosure, in FIGS. 14A-14B, two methods of applying a window method of the present disclosure to an FMCW system of detecting a fine movement of a target are presented. FIG. 14A shows a method of deriving both |Y(k)| and ∠Y(k) using a window method of the present disclosure, and FIG. 14B shows a method of deriving |Y(k)| using a conventional window method, and deriving ∠Y(k) using the window method of the present disclosure. That is, a profile |Y(k)| detected from a profile (Y(k)) computed through a fast Fourier transformer may be windowed by applying a conventional Blackman or Hamming window method.

Moreover, although there is no problem in detecting a fine movement using the window method in FIG. 14A, a conventional window method in FIG. 14B and a window method of the present disclosure may be used together in order to further improve the performance of detecting the position of a target. This is because the conventional window method has excellent characteristics of |Y(k)|, and the window method of the present disclosure has excellent characteristics of ∠Y(k).

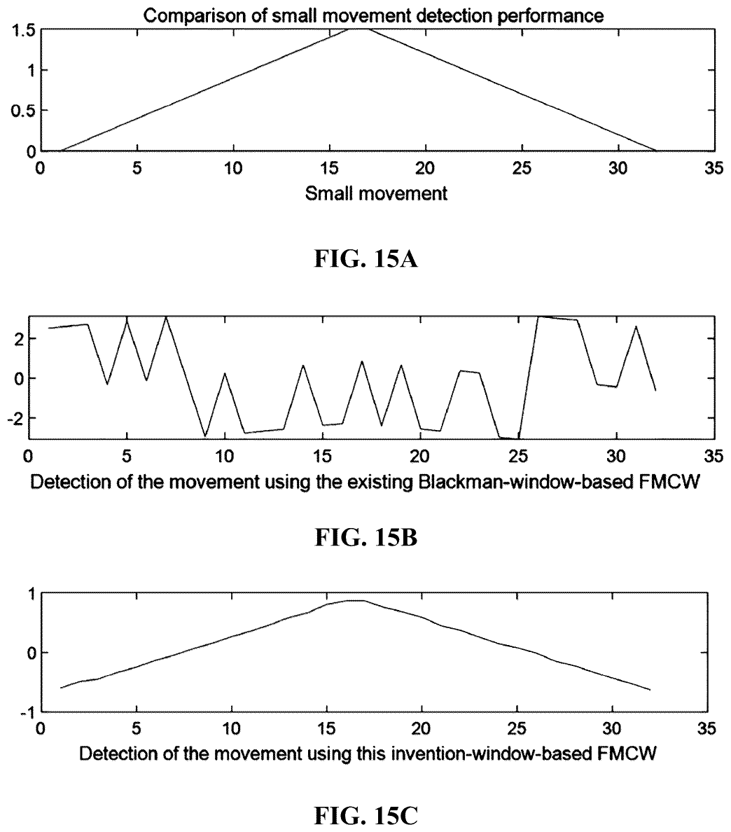

Next, in FIGS. 15A-15C, in a graph comparing the actual fine movement detection performance of a target, FIG. 15A is a waveform of an actual fine movement of a target. Here, the signal-to-noise ratio (SNR) was set to 20 dB. FIG. 15B is a result of showing a fine movement of a target detected by a conventional w_{Black}(n)-based FMCW system, and FIG. 15C is a result of a fine movement of a target detected by a w_{Black.new}(n)-based FMCW system of the present disclosure. As a result of comparing the graphs in FIGS. 15B-15C, a result detected by a w_{Black.new}(n)-based FMCW system in the present disclosure detects an actual fine movement waveform well, while a fine movement waveform detected by a conventional w_{Black}(n)-based FMCW system shows a result in which the fine movement cannot be properly detected due to the influence of noise.

As described above, according to a linearly continuous phased windowing method in a FMCW system for detection of bio-signals in the present disclosure, the present disclosure has an advantage of high detection performance and stability of a fine movement of a target compared to a conventional Hamming or Blackman window method.

Although the present disclosure has been shown and described in connection with specific embodiments in the above description, it will be readily apparent to anyone skilled in the art that various modifications and changes can be made without departing from the concept and scope of the disclosure as defined in the claims.

1. A linearly continuous phased windowing method in an FMCW system for detection of bio-signals, the method comprising:
(a) computing a profile W(k) by passing a complex window signal w(n) derived from the FMCW system through a fast Fourier transformer (FFT) to calculate an FFT(w(n)), and deriving a phase value ∠W(k) from the computed profile W(k);
(b) detecting a plurality of discontinuous points (k_{d} ld=0, . . . D−1) from the phase value ∠W(k) and then multiplying the corresponding profile W(k) by a frequency variation e^{jπ} to compute a new profile W_{new}(k);
(c) computing IFFT(^{w̅}_{new}(n)) by passing the new profile W_{new}(k) through an inverse fast Fourier transformer (IFFT) to calculate ^{w̅}_{new}(n); and
(d) computing real(w_{new}(n)) of a real part from the calculated ^{w̅}_{new}(n) to calculate w_{new}(n).

2. A linearly continuous phased windowing method in an FMCW system for detection of bio-signals, the method comprising:
(a) computing a profile W(k) by passing a complex window signal w(n) derived from the FMCW system through a fast Fourier transformer (FFT) to calculate an FFT(w(n)), and deriving a magnitude value |W(k)| from the computed profile W(k);
(b) computing a profile W(k) by passing a complex window signal w(n) derived from the FMCW system through a fast Fourier transformer (FFT) to calculate an FFT(w(n)), and deriving a phase value ∠W(k) from the computed profile W(k);
(c) detecting a plurality of discontinuous points (k_{d} ld=0, . . . D−1) from the phase value ∠W(k) and then multiplying the corresponding profile W(k) by a frequency variation e^{jπ} to compute a new profile W_{new}(k);
(d) computing IFFT(^{w̅}_{new}(n)) by passing the new profile W_{new}(k) through an inverse fast Fourier transformer (IFFT) to calculate ^{w̅}_{new}(n); and
(e) computing real(w_{new}(n)) of a real part from the calculated ^{w̅}_{ew}(n) to calculate w_{new}(n).
